(19) United States
(12) Patent Application Publication
(10) Pub. No.: US 2006/0292494 A1
(43) Pub. Date: Dec. 28, 2006

(54) Novel phthalocyanine derivatives, synthetic process thereof and their applications in optical recording media

(76) Inventor: Yen Cheng Tsai, Yonghe City (TW)

Correspondence Address: TROXELL LAW OFFICE PLLC, SUITE 1404, 5205 LEESBURG PIKE, FALLS CHURCH, VA, 22041, US

(21) Appl. No.: 11/158,071

(22) Filed: Jun. 22, 2005

(51) Int. Cl. G11B 7/24; C07D 487/22

(52) U.S. Cl. 430/270.16; 540/132

(57) ABSTRACT

An optical recording medium is disclosed, which is composed of a substrate, a recording layer comprising an organic dye upon which information can be recorded by a laser beam, a reflective layer and a protective layer formed in such order; whereas the aforementioned organic dye is a substituted phthalocyanine compound represented by formula (I): 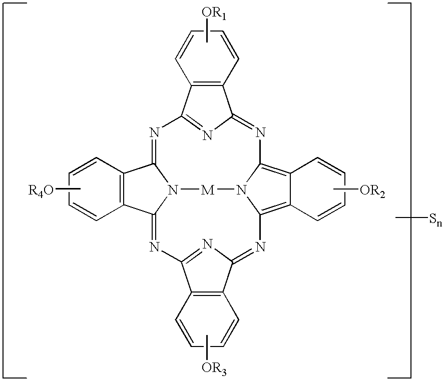
wherein R₁, R₂, R₃, and R₄ each independently represents an alkyl group having 1 to 12 carbon atoms and may be substituted by 0 to 6 halogen atoms, a hydroxyl group, an alkoxyl group having 1 to 6 carbon atoms, an alkylamino group having 1 to 6 carbon atoms, a dialkylamino group having 1 to 6 carbon atoms, or an alkylthio group having 1 to 6 carbon atoms; an alkenyl group having 2 to 12 carbon atoms; an alkynyl group having 2 to 12 carbon atoms; M may be two hydrogen atoms, a divalent metal, a monosubstituted trivalent metal, a disubstituted tetravalent metal, or an oxometal group; S is a moiety containing at least one —SO₃— group and n is an integer from 1 to 4.

### BACKGROUND OF THE INVENTION

1. Field of the Invention

This invention relates to a class of novel phthalocyanine derivatives as organic optical dyes and their applications in optical recording media, primarily to the use in recordable compact disc (CD-R).

2. Description of the Prior Art

Organic dyes have been widely employed in the field of optical recording of information. These recording media, which can only be recorded once but repeatedly played back, are therefore abbreviated as “WORM” (write once read many). Recordable compact discs, or the so-called CD-R, as the first example in disc format utilizing this technology, are known from “Optical Data Storage 1989,” Technical Digest Series, vol. 1, 45 (1989).

Among all the organic dyes for optical recording media, phthalocyanine derivatives are one of the most important categories, due largely to its high absorption in the near IR range (700˜900 nm). Compared to other organic dyes such as cyanines, phthalocyanine dyes exhibit better light-fastness and resistance to temperature and humidity.

Earlier literatures such as JP-A 154888 (1986), 197280 (1986), 246091 (1986), US 4769307 (1987) and JP-A 39388 (1988) described phthalocyanines as component materials in the optical recording layer of an optical recording medium. However, in terms of sensitivity, solubility, reflectivity, recording performance and other related physical properties, the above-described phthalocyanines could not be considered as appropriate materials for an optical recording medium.

In order to improve the aforementioned disadvantages associated with the use of phthalocyanine as an optical recording material, JP-A 62878 (1991) provided phthalocyanines with bulkier (greater steric hindrance) substituents on the phenyl rings. These materials, however, did not meet the recording requirements. In U.S. Pat. No. 5,229,507 (1993), phenyl-substituted phthalocyanines (also called naphthalocyanines) were proposed but the dyes exhibited insufficient solubility. Under certain process conditions, dyes would precipitate in the course of spin coating.

Solubility issue was further addressed in U.S. Pat. No. 5,641,879 (1997) by introducing various bulkier substituents onto the phenyl rings of phthalocyanine. However, inadequate reflective index was found. In U.S. Pat. No. 5,663,326 (1997), isomer effects on solubility were studied. It was reported that composition of the two isomers having one pair of alkoxy substituents heading toward each other needed to be greater than 80% in order to obtain desired solubility. It's obviously tedious for dye manufacturing processes and seemingly impractical to assure the isomer composition for quality control.

Another approach to address solubility issue was taken in U.S. Pat. No. 5,820,962 (1998) by introducing substituted trivalent metal as the central atom of phthalocyanine. Due to the bulkiness of the proposed structure, the compound dissolved well in polar solvents and the resulting discs showed good reflectivity. However, polar solvents inherited the hydrophilic character and inevitably led to the difficulties in absorbing moistures during recycling. Consequently, it resulted in quality inconsistency and even deterioration of the disc performances.

In addition to solubility, dye sensitivity is another critical factor for recording media, particularly to enable high-speed recording and fast access to the recorded information. Addition of the so-called “pit edge control agent” was proposed in U.S. Pat. No. 5,492,744 (1996) and JP-A-798887 to improve deviation and jitter properties. Ferrocenes and according derivatives (e.g. benzoylferrocene and n-butylferrocene) blended with substituted phthalocyanines at certain ratios were suggested. Pit formations were reported to be largely improved but material utilization had become an issue in the real-life practices. Since optical dyes account for considerable ratio in recordable disc cost structure, dyes (and dye solutions) have been designed and synthesized to be recycled. Phthalocyanine exhibits better solubility in the designated solvent (ethylcyclohexane, in this case) than ferrocene does. Consequently, the blended-in pit edge control agent tends to precipitate out during spin coating and recycling, resulting in the undesired concentration changes in the recycled dye solutions. The yield (productivity) was inferior to those with single dyes. A minor modification was seen in U.S. Pat. No. 5,789,138 (1998), in which phthalocyanine was blended with (or dissolved in) melted additive (e.g. benzimidazole) so that coordination occurred from the additive to the center metal of phthalocyanine. The thus-obtained dyestuff would exhibit better intermolecular associations to obtain desired film patterns. However, the trade-off between the limited coordination chemistry and the corresponding dye performance made it difficult to optimize disc performances.

Halogenation on phthalocyanine was also reported to improve sensitivity. U.S. Pat. No. 5,646,273 (1997) claimed OPC (optimal power calibration, or “optimal recording power”) was effectively improved by halogenation on the alkyl and/or alkoxy substituents to phthalocyanine. Halogenation directly on the phenyl rings of phthalocyanine, on the other hand, was also proposed in U.S. Pat. No. 6,087,492 (2000). However, these resulting discs still showed insufficient sensitivity and unsatisfactory controls in the formation of information pits. Nevertheless, precise reaction control in the degree of halogenation was difficult. The resulting compound was inevitably a mixture containing various numbers of halogen atoms, leading to unstable dye quality and inconsistent disc properties.

In U.S. Pat. No. 6,087,492 (2000), substituted phthalocyanine with divalent metal as the central atom was formylated, further reduced, followed by esterification. Without pit edge control agent in the structure or blended in the formula as described in U.S. Pat. No. 5,492,744 (1996), the resulting dye did not render satisfactory properties. Improvement was made in U.S. Pat. No. 6,399,768 B1 (2002) and U.S. Pat. No. 6,790,593 B2 (2004) by chemically bonding ferrocene to phthalocyanine through ester linkage. These metallocenyl phthalocyanines were halogenated (mainly brominated) at various degrees, depending on the central metal atoms. It was claimed that the resulting dyes exhibited good optical sensitivity and solubility to solvents such as di-butyl ether (DBE) and ethylcyclohexane (ECH). Although these dyes exhibited good recording properties, their syntheses were relatively complicated for industrial processes. Not to mention the high raw material (especially, the metallocences) costs. As the CD-R market is seemingly saturated, such high cost dyes can not meet the stringent demand for an inexpensive but good CD-R product.

In order to fulfill the different requirements from various recorders, and most importantly, to significantly lower the dye cost, this invention provides innovative optical dyes by incorporating sulfonate compounds in place of the traditional ferrocenyl groups onto the phthalocyanine structures. Discs made of these novel optical dyes show excellent performances at 1× through 52× recordings.

### SUMMARY OF THE INVENTION

One of the major objects of this invention is to provide a novel organic optical dye comprising a sulfonate group chemically bonded to an unsubstituted or substituted phthalocyanine. The resulting compound can be represented by formula (I): 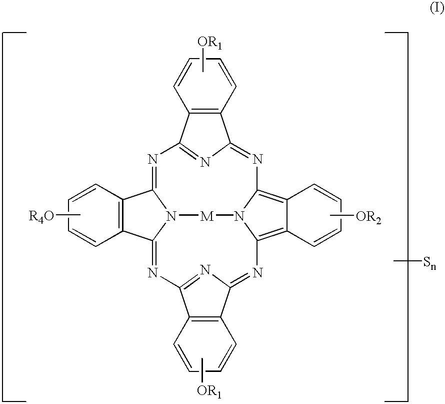
wherein R₁, R₂, R₃, and R₄ each independently represents an alkyl group having 1 to 12 carbon atoms and may be substituted by 0 to 6 halogen atoms, a hydroxyl group, an alkoxyl group having 1 to 6 carbon atoms, an alkylamino group having 1 to 6 carbon atoms, a dialkylamino group having 1 to 6 carbon atoms, or an alkylthio group having 1 to 6 carbon atoms; an alkenyl group having 2 to 12 carbon atoms; an alkynyl group having 2 to 12 carbon atoms; M may be two hydrogen atoms, a divalent metal, a monosubstituted trivalent metal, a disubstituted tetravalent metal, or an oxometal group; S is a moiety containing at least one —SO₃— group and n is an integer from 1 to 4.

Another object of this invention is to provide the syntheses of the organic dyes of formula (I).

Yet another object of this invention is to provide an organic dye of formula (II):

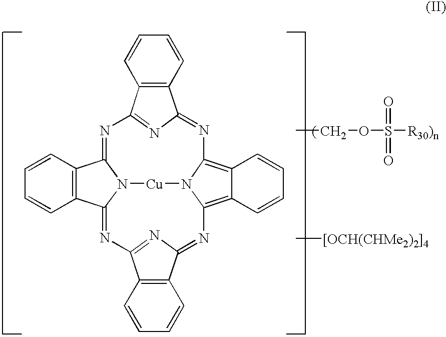

wherein R₃₀ is a trifluoromethyl group or a tolyl group, and n is an integer from 1 to 4.

Still another object of this invention is to provide the syntheses of the organic dyes of formula (II).

Another object of this invention is to provide the use of said organic dyes of formula (I) or (II) as the optical dyes in the recording layer of a recordable disc to impart the recordable discs excellent recording properties.

Yet another object of this invention is to provide an optical recording medium comprising said novel phthalocyanine derivatives of formula (I) or (II) as the optical dyes in the recording layer thereof.

### DETAILED DESCRIPTION OF THE PREFERRED EMBODIMENTS

As previously described, this invention provides phthalocyanine derivatives as optical dyes to be used in the recording layer of an optical recording medium composed of a pre-grooved substrate, a recording layer upon which information can be recorded by a laser beam, a reflective layer and a protective layer formed in such order. Said optical dyes are phthalocyanine derivatives (or mixture of derivatives) whose structures can be represented by following formula: 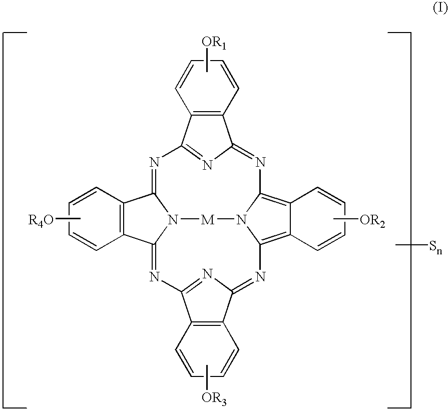
wherein R₁, R₂, R₃, and R₄ each independently represents an alkyl group having 1 to 12 carbon atoms and may be substituted by 0 to 6 halogen atoms, a hydroxyl group, an alkoxyl group having 1 to 6 carbon atoms, an alkylamino group having 1 to 6 carbon atoms, a dialkylamino group having 1 to 6 carbon atoms, or an alkylthio group having 1 to 6 carbon atoms; an alkenyl group having 2 to 12 carbon atoms; an alkynyl group having 2 to 12 carbon atoms; M may be two hydrogen atoms, a divalent metal, a monosubstituted trivalent metal, a disubstituted tetravalent metal, or an oxometal group; S is a moiety containing at least one —SO₃— group and n is an integer from 1 to 4.

The phthalocyanine derivative employed in this invention can be prepared according to a process described in, for example, EP 703280, or it can be obtained from commercial sources. Among those phthalocyanine derivatives, the α-substituted phthalocyanines are most preferred. Those phthalocyanine derivatives used in this invention may comprise several isomers as represented by formulae (2) to (5): 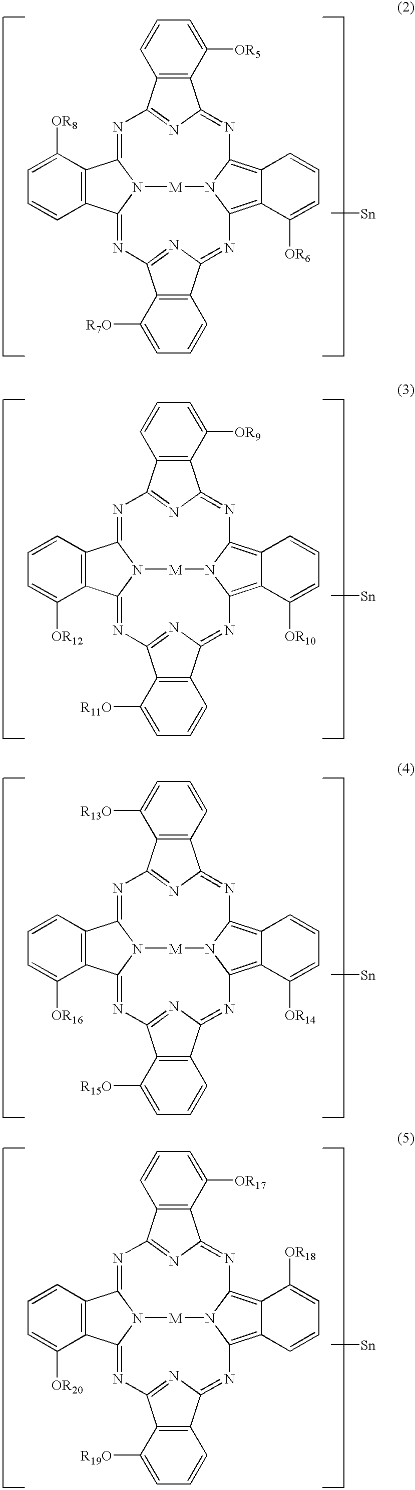
wherein R₅ to R₂₀ each independently represents an alkyl group having 1 to 12 carbon atoms and may be substituted by 0 to 6 halogen atoms, a hydroxyl group, an alkoxyl group having 1 to 6 carbon atoms, an alkylamino group having 1 to 6 carbon atoms, a dialkylamino group having 1 to 6 carbon atoms, or an alkylthio group having 1 to 6 carbon atoms; an alkenyl group having 2 to 12 carbon atoms; an alkynyl group having 2 to 12 carbon atoms; M may be two hydrogen atoms, a divalent metal, a monosubstituted trivalent metal, a disubstituted tetravalent metal, or an oxometal group.

The isomeric composition of the aforementioned four α-substituted phthalocyanines may vary according to reaction conditions and as desired. Preferred substituents therein are secondary alkyl, alkenyl, or alkynyl groups. The most preferred substituents are alkyl, alkenyl, or alkynyl groups containing 2 to 4 secondary, tertiary, or quaternary carbon atoms.

As R₁ to R₂₀ in formulae (1) to (5), representative alkyl groups are, for example, methyl, ethyl, n-propyl, i-propyl, n-butyl, i-butyl, s-butyl, t-butyl, n-pentyl, i-pentyl, cyclopentyl, 2-methylbutyl, 1,2-dimethylpropyl, n-hexyl, cyclohexyl, 2-methylpentyl, 3-methylpentyl, 4-methylpentyl, 1,2-dimethylbutyl, 1,3-dimethylbutyl, 2,2-dimethylbutyl, 2,3-dimethylbutyl, 3,3-dimethylbutyl, 1-(i-propyl)propyl, n-heptyl, cycloheptyl, 2-methylhexyl, 3-methylhexyl, 4-methylhexyl, 5-methylhexyl, 1,2-dimethylpentyl, 1,3-dimethylpentyl, 1,4-dimethylpentyl, 2,2-dimethylpentyl, 2,3-dimethylpentyl, 2,4-dimethylpentyl, 1-ethyl-3-methylbutyl, 2-(i-propyl)butyl, 2-methyl-1-(i-isopropyl)propyl, n-octyl, cyclooctyl, 2-ethylhexyl, 3-methyl-1-(i-isopropyl)butyl, 2-methyl-1-(i-isopropyl)butyl, 1-t-butyl-2-methylpropyl, n-nonyl, cyclononyl, n-decyl, cyclodecyl, undecyl, dodecyl; preferable substituents are branched alkyl groups containing 2 to 4 secondary, tertiary, or quaternary carbon atoms, for example, i-propyl, i-butyl, s-butyl, t-butyl, i-pentyl, 2-methylbutyl, 1,2-dimethylpropyl, 1,3-dimethylbutyl, 1-(i-propyl)propyl, 1,2-dimethylbutyl, 1,4-dimethylpentyl, 2-methyl-1-(i-propyl)propyl, 1-ethyl-3-methylbutyl, 2-ethylhexyl, 3-methyl-1-(i-propyl)butyl, 2-methyl-1-(i-propyl)butyl, 1-t-butyl-2-methylpropyl, 2,4-dimethyl-3-pentyl; most preferable substituents are, for example, 1-t-butyl-2-methylpropyl, 2-methyl-1-isopropylbutyl, 2,4-dimethyl-3-pentyl.

Representative halogenated alkyl groups are, for example, chloromethyl, 1,2-dichloroethyl, 1,2-dibromoethyl, 2,2,2-trifluoroethyl, 2,2,2-trichloroethyl, 2,2,2-tribromoethyl, 1,1,2,2,2-pentachloroethyl, 1,1,1,3,3,3-hexafluoro-2-propyl.

Representative hydroxyalkyl groups are, for example, hydroxymethyl, 2-hydroxyethyl, 1,2-dihydroxyethyl, 3-hydroxypropyl, 2,3-dihydroxypropyl, 3-hydroxybutyl, 4-hydroxybutyl, 3-hydroxypentyl, 4-hydroxypentyl, 5-hydroxypentyl, 2-hydroxyhexyl, 3-hydroxyhexyl, 4-hydroxyhexyl, 5-hydroxyhexyl, 6-hydroxyhexyl, hydroxyheptyl, hydroxyoctyl, hydroxynonyl, hydroxydecyl, hydroxyundecyl, hydroxydodecyl.

Representative alkoxyalkyl groups are, for example, methoxymethyl, methoxyethyl, methoxypropyl, methoxybutyl, methoxypentyl, methoxyhexyl, 3-methoxycycolpentyl, 4-methoxycyclohexyl, ethoxyethyl, ethoxypropyl, ethoxybutyl, ethoxypentyl, ethoxyhexyl, 4-ethoxycyclohexyl, propoxyethyl, propoxypropyl, propoxybutyl, propoxypentyl, propoxyhexyl, butoxyethyl, butoxypropyl, butoxybutyl, 1,2-dimethoxyethyl, 1,2-diethoxyethyl, 1,2-dimethoxypropyl, 2,2-dimethoxypropyl, diethoxybutyl and butoxyhexyl; preferable substituents are alkoxyalkyl groups containing 2 to 10 carbon atoms, for example, methoxymethyl, methoxyethyl, ethoxypropyl, ethoxybutyl, propoxyhexyl, 1,2-dimethoxypropyl, 2,2-dimethoxypropyl, diethoxybutyl and butoxyhexyl; most preferable substituents are alkoxyalkyl groups containing 2 to 6 carbon atoms, for example, methoxymethyl, methoxyethyl, ethoxypropyl, ethoxybutyl.

Representative alkylaminoalkyl groups are, for example, methylaminomethyl, methylaminoethyl, methylaminopropyl, methylaminobutyl, ethylaminoethyl, ethylaminopropyl, ethylaminobutyl, ethylaminopentyl, ethylaminohexyl, ethylaminoheptyl, ethylaminooctyl, propylaminoethyl, propylaminopropyl, propylaminobutyl, propylaminopentyl, propylaminohexyl, i-propylaminoethyl, i-propylaminopropyl, i-propylaminobutyl, i-propylaminopentyl, i-propylaminohexyl, butylaminoethyl, butylaminopropyl, butylaminopentyl, butylaminohexyl; preferable substituents are alkylaminoalkyl groups containing 2 to 8 carbon atoms, for example, methylaminomethyl, methylaminoethyl, ethylaminopropyl, ethylaminobutyl, ethylaminopentyl, ethylaminohexyl, propylaminobutyl, propylaminopentyl; most preferable substituents are, for example, alkylaminoalkyl groups containing 2 to 6 carbon atoms, for example, methylaminomethyl, methylaminoethyl, ethylaminopropyl, ethylaminobutyl.

Representative dialkylaminoalkyl groups are, for example, dimethylaminomethyl, dimethylaminoethyl, dimethylaminopropyl, dimethylaminobutyl, diethylaminoethyl, diethylaminopropyl, diethylaminobutyl, diethylaminopentyl, diethylaminohexyl, diethylaminoheptyl, diethylaminooctyl, dipropylaminoethyl, dipropylaminopropyl, dipropylaminobutyl, dipropylaminopentyl, dipropylaminohexyl, di(i-propyl)aminoethyl, di(i-propyl)aminopropyl, di(i-propyl)aminobutyl, di(i-propyl)aminopentyl, di(i-propyl)aminohexyl; preferable substituents are dialkylaminoalkyl groups containing 2 to 10 carbon atoms, for example, dimethylaminomethyl, dimethylaminoethyl, diethylaminopropyl, diethylaminobutyl, diethylaminopentyl, diethylaminohexyl; most preferable substituents are dialkylaminoalkyl groups containing 2 to 6 carbon atoms, for example, dimethylaminomethyl, dimethylaminoethyl, diethylaminoethyl.

Representative alkylthioalkyl groups are, for example, methylthiomethyl, methylthioethyl, methylthiopropyl, methylthiobutyl, methylthiopentyl, methylthiohexyl, 3-methylthiocycolpentyl, 4-methylthiocyclohexyl, ethylthioethyl, ethylthiopropyl, ethylthiobutyl, ethylthiopentyl, ethylthiohexyl, 4-ethylthiocyclohexyl, propylthiobutyl, propylthiopentyl, propylthiohexyl; preferable substituents are alkylthioalkyl groups containing 2 to 8 carbon atoms, for example, methylthiomethyl, methylthioethyl, ethylthiopropyl, ethylthiobutyl, propylthiohexyl; most preferable substituents are alkylthioalkyl groups containing 2 to 6 carbon atoms, for example, methylthiomethyl, methylthioethyl, ethylthiopropyl, ethylthiobutyl.

Representative alkenyl groups are, for example, ethenyl, n-propenyl, i-propenyl, n-butenyl, i-butenyl, s-butenyl, n-pentenyl, i-pentenyl, cyclopentenyl, 2-methylbutenyl, 1,2-dimethylpropenyl, n-hexenyl, cyclohexenyl, n-heptenyl, cycloheptenyl, n-octenyl, cyclooctenyl, n-nonenyl, cyclononenyl, n-decenyl, cyclodecenyl, undecenyl and dodecenyl; preferable substituents are alkenyl groups containing 2 to 6 carbon atoms, for example, ethenyl, n-propenyl, i-propenyl, n-butenyl, i-butenyl, s-butenyl, n-pentenyl, i-pentenyl, cyclopentenyl, 2-methylbutenyl, 1,2-dimethylpropenyl, n-hexenyl, cyclohexenyl; most preferable substitutents are alkenyl groups containing 2 to 4 carbon atoms, for example, ethenyl, n-propenyl, i-propenyl, n-butenyl, i-butenyl, s-butenyl, t-butenyl.

Representative alkynyl groups are, for example, ethynyl, propynyl, n-butynyl, s-butynyl, n-pentynyl, i-pentynyl, cyclopentynyl, 2-methylbutynyl, n-hexynyl, cyclohexynyl, n-heptynyl, cycloheptynyl, n-octynyl, cyclooctynyl, n-nonynyl, cyclononynyl, n-decynyl, cyclodecynyl, undecynyl, dodecynyl; preferable substituents are alkynyl groups containing 2 to 6 carbon atoms, for example, ethynyl, propynyl, n-butynyl, s-butynyl, n-pentynyl, i-pentynyl, cyclopentynyl, 2-methylbutynyl, n-hexynyl, cyclohexynyl; most preferable substituents are alkynyl groups containing 2 to 4 carbon atoms, for example, ethynyl, propynyl, n-butynyl, s-butynyl.

Representative divalent central metal M in formulae (1) to (5) may be, for example, copper, zinc, iron, cobalt, nickel, palladium, platinum, manganese, tin, ruthenium, osmium; most preferred metals are copper, cobalt, nickel, palladium, platinum. Representative monosubstituted trivalent metals are, for example, fluorine-aluminum, chlorine-aluminum, bromine-aluminum, iodine-aluminum, fluorine-indium, chlorine-indium, bromine-indium, iodine-indium, fluorogallium, chlorogallium, bromogallium, iodogallium, fluorothallium, chlorothallium, bromothallium, iodothallium, hydroxyaluminum, hydroxymanganese. Representative disubstituted tetravalent metals are, for example, difluorosilicon, dichlorosilicon, dibromosilicon, diiodosilicon, difluorotin, dichlorotin, dibromotin, diiodotin, difluorogermanium, dichlorogermanium, dibromogermanium, diiodogermanium, difluorotitanium, dichlorotitanium, dibromotitanium, diiodotitanium, dihydroxysilicon, dihydroxytin, dihydroxygermanium, dihydroxymanganese. Representative oxometal groups are, for example, oxovanadium, oxomanganese, oxotitanium.

To enhance the performances of phthalocyanine during recording, according to this invention, a sulfonate compound is covalently bonded to a substituted or unsubstituted phthalocyanine derivative. Not only does the thus-obtained dye exhibit good reflectivity and sensitivity, it can also effectively lower the optimal recording power to meet the requirements for 1× to 52× recordings on various writers.

There are many routes to covalently link a sulfonate compound to a substituted or un-substituted phthalocyanine. Typical methods are reacting a hydroxylated phthalocyanine with trifluoromethanesulfonic anhydride under proper conditions, or reacting a hydroxylated phthalocyanine with p-toluenesulfonyl chloride in the presence of an organic base (e.g. pyridine) as the catalyst. The thus-obtained compound can be represented by formula (6):

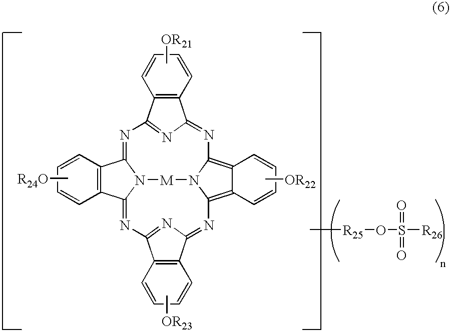

In a preferred embodiment, the phthalocyanine derivative of formula (6) comprises a compound wherein R₂₁, R₂₂, R₂₃ and R₂₄ are 2,4-dimethyl-3-pentyl, R₂₅=—CH₂—, and M═Cu, that is, a compound represented by formula (II): 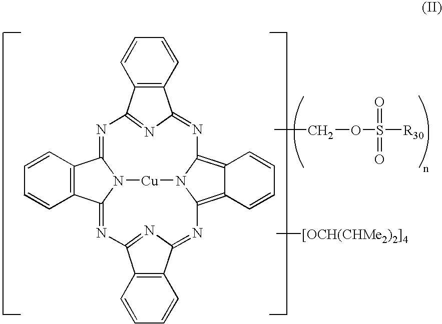
wherein R₃₀ is a trifluoromethyl group or a tolyl group, n is an integer from 1 to 4.

Yet, in another preferred embodiment, the phthalocyanine derivative of formula (6) comprises a compound wherein R₂₁, R₂₂, R₂₃ and R₂₄ are 2,4-dimethyl-3-pentyl, M=Cu, R₂₅=—CH₂—, and R₂₆=—CF₃, and can be prepared by the process described above. More specifically, hydroxymethylated tetra-α-(2,4-dimethyl-3-pentoxy)copper phthalocyanine reacts with trifluoromethanesulfonic anhydride under neat condition at low temperature to yield a product of formula (7):

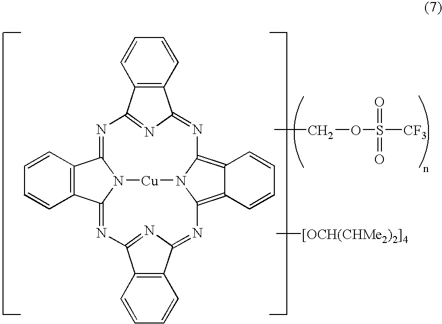

In still another embodiment, hydroxymethylated tetra-α-(2,4-dimethyl-3-pentoxy)copper phthalocyanine and p-toluenesulfonyl chloride are dissolved in a suitable solvent, for example, dichloromethane, and the resulting solution is allowed to react in the presence of an organic base such as pyridine as the catalyst, at room temperature, to yield a product of formula (8):

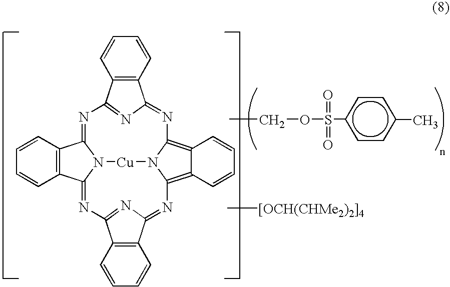

Another synthetic route to chemically bond a sulfonate compound to a substituted or unsubstituted phthalocyanine is to react phthalocyanine sulfonyl chloride with an alcohol to form a sulfonate compound that can be represented by formula (9):

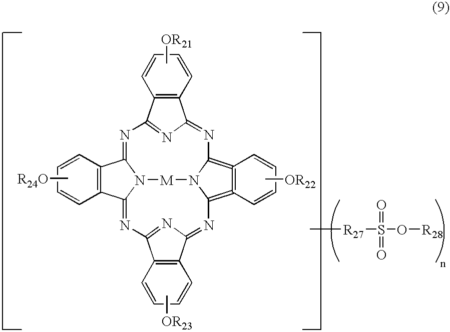

R₂₁˜R₂₄ and M in formulae (6) and (9) are defined as what R₁˜R₂₀ and M are in formulae (1) to (5); R₂₅ and R₂₇ may represent independently a single bond, —CH₂—, —CH₂CH₂—, —CH═CH—, —CH₂—C(═O)—, —CH₂—CH₂—C(═O)—, —O—R₂₉—, —C(═O)—O—R₂₉— or —O—(C═O)—R₂₉—, wherein R₂₉ is a C₁-C₄ alkylene group or a C₂-C₄ alkenylene group; preferably, R₂₅ and R₂₇ are —CH₂— and —C(═O)—O—R₂₉—; R₂₆ and R₂₈ are defined as what R₁˜R₂₀ are in formulae (1) to (5), and preferably a methyl or a trifluoromethyl group; a C₆-C₁₈ aryl group such as phenyl, naphthyl, biphenyl, anthryl, phenanthryl, or terphenylyl, preferably a phenyl group; or a C₇-C₁₈ aralkyl group, for example, —(CH₂)₃₋₁₂-phenyl group or -phenylene-(CH₂)₃₋₁₁—CH₃, preferably a tolyl group; and n is an integer from 1 to 4.

Another preferred embodiment of this invention relates to an optical recording medium composed of a substrate, a recording layer, a reflective layer and a protective layer formed in such order, wherein said recording layer comprises the aforementioned phthalocyanine derivatives provided by this invention as the optical dyes.

In said optical recording medium provided by this invention, the substrate is generally made of an optical transparent resin, such as an acrylic resin, polyethylene resin, polystyrene resin or polycarbonate resin. Meanwhile, the surface of the substrate can be treated with a thermosetting resin or UV cross-linkable resin, if necessary.

The recording layer can be formed by spin coating a solution of the phthalocyanine derivatives provided by this invention onto the substrate. The spin coating process can be carried out as follows: dissolving the phthalocyanine derivatives of this invention in a solvent at an appropriate ratio, desirably no more than 5% wt/vol (weight/volume ratio), preferably 1.5-3%. Subsequently, the resulting solution can be applied onto the substrate via conventional spin coating technique. The thickness of a recording layer is generally between 50 and 300 nm, preferably between 80 and 150 nm.

Taking into account the solubility of organic optical dye in a specific solvent and possible erosion toward substrate by the solvent, a preferred solvent for spin coating could be selected from halogenated hydrocarbons, for example, dichloromethane, chloroform, carbon tetrachloride, trichloroethane, dichloroethane, tetrachloroethane and dichlorodifluoroethane; ethers, for example, ethyl ether, propyl ether, butyl ether and cyclohexyl ether; alcohols, for example, methanol, ethanol, propanol, tetrafluoropropanol and butanol; ketones, for example, acetone, trifluoroacetone, hexafluoroacetone and cyclohexanone; and hydrocarbons, for example, hexane, cyclohexane, methylcyclohexane, dimethylcyclohexane, ethylcyclohexane, octane and cyclooctane.

The reflective layer is composed mainly of metals such as copper, aluminum, gold or silver, or alloy thereof. The reflective layer can be formed by depositing suitable material(s) upon the recording layer through vacuum deposition or sputtering at a thickness between 1 and 200 nm.

The protective layer is composed mostly of a thermosetting resin or a UV cross-linkable resin, preferably a transparent resin. In common practice, the protective layer can be formed by spin coating the resin onto the reflective layer to form a layer with thickness between 0.1 and 500 micrometer, preferably between 0.5 and 50 micrometer.

From the practicality viewpoint, polycarbonate resin is nowadays predominantly employed as the substrate material of the optical recording medium while spin coating process is the primary choice for forming the recording and protective layers.

The prime spirit of this invention can be best illustrated, but not limited to, in further detail by the following examples. Any directly or indirectly related derivatives based upon the prime spirit of this invention will be considered to fall within the scope of the invention.

### EXAMPLES

### Example 1

10.0 g tetra-α-(2,4-dimethyl-3-pentoxyl)copper phthalocyanine derivative (prepared according to EP 703280) was weighed into a 250 ml round-bottom flask with nitrogen purge. 50 ml toluene and 5.4 g N-methylformamide were then added thereto. After complete dissolution, the temperature of the resulting solution was lowered to 0° C. Once the temperature was stabilized, 5.6 g POCl₃ was slowly added into the reaction solution, while keeping the temperature not exceeding 5° C. The cooling system was removed after the complete addition of POCl₃ and the temperature was further raised to 50° C. The reaction solution was stirred at 50° C. for 24 hours. Reaction was monitored with thin layer chromatography (TLC) till completion. The reaction mixture was then poured into iced 200 ml sodium acetate (41.5 g) solution and stirred for 30 minutes, followed by extraction with 100 ml×3 toluene. The combined organic layers were dried over 20 g anhydrous magnesium sulfate which was later filtered off, followed by concentrating to about 60 ml under reduced pressure. The concentrate was then poured into 1 L mixed solvents of methanol/water (98/2), vigorously stirred for 30 minutes. Product was collected by filtration, followed by washing with 1 L methanol, and dried in a vacuum oven at 70° C. for two days. The thus-obtained green powder was 9.5 g (64% theory).

| | | | | |
|---|---|---|---|---|
| Elemental analysis: | found (%): | C: 69.21 | H: 6.79 | N: 10.44 |
| | Calculated (%): | C: 69.06 | H: 6.84 | N: 10.56 |

UV-VIS (DBE): λ max = 710 nm
IR (KBr): C═O absorption at 1675 cm⁻¹

### Example 2

1.03 g sodium borohydride was weighed into a 250 ml three-necked round-bottom flask with nitrogen purge, followed by addition of 40 ml ethanol to dissolve the sodium borohydride. 10.0 g formylated tetra-α-(2,4-dimethyl-3-pentoxyl)copper phthalocyanine (as prepared in Example 1) was dissolved in 40 ml tetrahydrofuran (THF), and was subsequently added into the reducing agent solution prepared above. The resulting reaction solution was stirred vigorously at ambient temperature for 24 hours and was monitored with TLC. At the end of the reaction, the insoluble was filtered off and the reaction was terminated by pouring 200 ml 20% saline solution thereto. The mixture was then extracted with 40 ml×3 toluene. The combined organic layers were dried over 20 g anhydrous magnesium sulfate which was later filtered off, followed by concentrating to about 40 ml under reduced pressure. The concentrate was then poured into 1L mixed solvents of methanol/water (98/2), stirred vigorously for 30 minutes. Product was collected by filtration, followed by washing with IL methanol, and dried in a vacuum oven at 70° C. for two days. The thus-obtained green powder was 9.4 g (95% theory).

| | | | | |
|---|---|---|---|---|
| Elemental analysis: | found (%): | C: 68.77 | H: 7.20 | N: 10.56 |
| | Calculated (%): | C: 68.93 | H: 7.02 | N: 10.54 |

UV-VIS (DBE): λ max = 713.5 nm
IR (KBr): C═O absorption at 1675 cm⁻¹ disappeared, and OH absorption appeared at 3210 cm⁻¹

### Example 3

10.0 g hydroxymethylated tetra-α-(2,4-dimethyl-3-pentoxy)copper phthalocyanine (as prepared in Example 2) was weighed into a 250 ml reactor, 40 ml toluene was then added therein under stirring with nitrogen purge. After complete dissolution, the temperature of the resulting solution was lowered to 0° C. 48.64 ml solution of trifluoromethanesulfonic anhydride in toluene (6.25%) was slowly added therein, while keeping the temperature of the reaction solution not exceeding 5° C. The cooling system was removed after complete addition of the anhydride solution so that the temperature was raised to room temperature. The solution was further stirred at room temperature for 2 hours and the reaction was monitored with thin layer chromatography (TLC) till completion. The reaction was terminated by pouring the reaction mixture into mixed solvents of methanol/water (80 ml/240 ml) under stirring for 30 minutes, followed by extraction with 100 ml×3 toluene. The combined organic layers were dried over 20 g anhydrous magnesium sulfate which was later filtered off, followed by concentrating to about 60 ml under reduced pressure. The concentrate was then poured into IL mixed solvents of methanol/water (98/2) and vigorously stirred for 30 minutes. Product was collected by filtration, washed with IL methanol, and dried in a vacuum oven at 70° C. for two days to yield 9.4 g green powder (83% theory).

| | | | | |
|---|---|---|---|---|
| Elemental analysis: | found (%): | C: 62.50 | H: 6.18 | N: 9.08 |
| | calculated (%): | C: 62.32 | H: 6.16 | N: 9.38 |

UV-VIS(DBE): λ max = 714.5 nm
IR (KBr): SO₃ at 1367, 1152 cm⁻¹
TGA: main decomposition temperature started at 253° C. (˜37%)

### Example 4

3.4 g p-toluenesulfonyl chloride was weighed into a 250 ml round bottom three-neck flask, 10 ml dichloromethane was added thereto under nitrogen purge, and the resulting mixture was stirred for 30 minutes. Thereafter, 20 ml pyridine was slowly added to the reaction mixture and stirred for additional 20 minutes. Separately, a solution containing 10.0 g hydroxymethylated tetra-α-(2,4-dimethyl-3-pentoxy)copper phthalocyanine (prepared in example 2) and 15 ml dichloromethane was prepared and then added into the previous 250 ml flask to react at room temperature for 12 hours. The reaction was terminated by pouring the reaction mixture into 300 ml water and stirred for about 30 minutes, followed by extraction with 100 ml×3 toluene. The combined organic layers were dried over 20 g anhydrous magnesium sulfate which was later filtered off, followed by concentrating to about 60 ml under reduced pressure. The concentrate was then poured into one liter n-hexane with vigorously stirring for 30 minutes. Product was collected by filtration and dried in a vacuum oven at 70° C. for two days to yield 9.0 g green powder (80% theory).

| | | | | |
|---|---|---|---|---|
| Elemental analysis: | found (%): | C: 67.45 | H: 6.25 | N: 9.50 |
| | calculated (%): | C: 67.11 | H: 6.63 | N: 9.21 |

UV-VIS(DBE): λ max = 714.0 nm
IR (KBr): SO₃ at 1367, 1152 cm⁻¹
TGA: main decomposition started at 227° C. (˜35%)

### Example 5

A dye solution was so prepared that compound of Example 3 was dissolved in the mixed solvents of dibutyl ether (DBE) and 2,6-dimethyl-4-heptanone (95/5, vol/vol) to form a 2.8% wt/vol (solute weight/solvent volume) solution. After vigorously stirred for 1 hour, the solution was first filtered through a Teflon filter of 0.2 micrometer pore size and then spin-coated onto a 1.2 mm-thick pre-grooved disc (average groove depth=195 nm, average groove width=600 nm, and track pitch=1.7 micrometer) at an initial rotation speed of 400 rpm. The rotation was further raised to 300 rpm to remove excess solution. The thus-formed homogeneous recording layer was dried in circulating hot air at 60° C. for 15 minutes. Subsequently, a 60 nm-thick silver reflective layer was sputtered upon the recording layer in a vacuum sputtering apparatus (ALCATEL, ATP150). Finally, a UV hardener (ROHM AND HAAS DEUTSCHLAND GMBH, Rengolux 3203-031v6 clear-CD LACQUER) was spin-coated over the silver reflective layer and further subject to UV curing to form a protective layer with a thickness of 5 mm. Information was recorded successively over the thus-produced blank CD-R disc at 52× recording speed on a commercial writer (Liteon LTR-52327S). The recorded disc was then tested with an automatic compact disc testing system (Pulstec OMT-2000×4) to measure the signals at 1×. Major data at 40-minute position were compiled in Table (1).

### Example 6

The procedures described in Example 5 were repeated. Information was recorded successively over the thus-produced blank CD-R disc at 52× recording speed on a commercial writer (Liteon LTR-52327S). The recorded disc was then tested with an automatic compact disc testing system (Pulstec OMT-2000×4) to measure the signals at 1×. Major data at 75-minute position were compiled in Table (1).

TABLE (1)

| Position | I3 | I11 | Rtop | BLER | Jit3T | Jit11T |
|---|---|---|---|---|---|---|
| 40-min | 0.31 | 0.69 | 0.63 | 3.1 | 25 | 27 |
| 75-min | 0.30 | 0.66 | 0.63 | 2.4 | 28 | 31 |

| | |
|---|---|
| (A) I3 | Modulation at 3T(T = 231.4 ns) |
| (B) I11 | Modulation at 11T(T = 231.4 ns) |
| (C) Rtop | Reflectivity |
| (D) BLER | Block Error Rate |
| (E) Jit3T | Jitter value at 3T |
| (F) Jit11T | Jitter value at 11T |

### Example 7

A dye solution was so prepared that compound of Example 4 was dissolved in the mixed solvents of 1-propanol and 4-hydroxy-4-methyl-2-pentanone (95:5) to form a 1.7% wt/vol (solute weight/solvent volume) solution. After vigorously stirred for 1 hour, the solution was first filtered through a Teflon filter of 0.2 micrometer pore size and then spin-coated onto a 1.2 mm-thick pre-grooved disc (average groove depth=195 nm, average groove width=600 nm, and track pitch=1.7 micrometer) at an initial rotation speed of 400 rpm. The rotation was further raised to 3000 rpm to remove excess solution. The thus-formed homogeneous recording layer was dried in circulating hot air at 60° C. for 15 minutes. Subsequently, a 60 nm-thick silver reflective layer was sputtered upon the recording layer in a vacuum sputtering apparatus (ALCATEL, ATP150). Lastly, a UV hardener (ROHM AND HAAS DEUTSCHLAND GMBH, Rengolux 3203-031v6 clear-CD LACQUER) was spin-coated over the silver reflective layer and further subject to UV curing to form a protective layer with a thickness of 5 mm. Information was recorded successively over the thus-produced blank CD-R disc at 52× recording speed on a commercial writer (BenQ CD-RW 5232×). The recorded disc was then tested with an automatic compact disc testing system (Pulstec OMT-2000×4) to measure the signals at 1×. Major data at 40-minute position were compiled in Table (2).

### Example 8

The procedures described in Example 7 were repeated. Information was recorded successively over the thus-produced blank CD-R disc at 52× recording speed on a commercial recorder (BenQ CD-RW 5232×). The recorded disc was then tested with an automatic compact disc testing system (Pulstec OMT-2000×4) to measure the signals at 1×. Major data at 75-minute position were compiled in Table (2).

TABLE (2)

| Position | I3 | I11 | Rtop | BLER | Jit3T | Jit11T |
|---|---|---|---|---|---|---|
| 40-min | 0.31 | 0.70 | 0.62 | 5.2 | 29 | 27 |
| 75-min | 0.30 | 0.68 | 0.61 | 4.5 | 30 | 31 |

As the data shown in Tables (1) and (2), it is evident that the optical recording medium utilizing the invented phthalocyanine dyes all exhibit excellent performances at different recording speeds on various commercial writers. In addtion, the performances of the recroded discs meet the specifications as defined in the Orange Book.

1. An optical recording material with structure represented by formula I 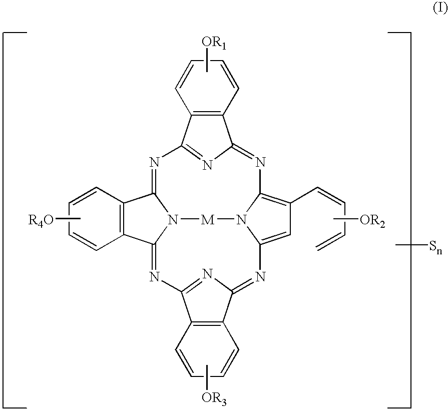
wherein
each R₁, R₂, R₃, and R₄ may be independently an alkyl group having 1 to 12 carbon atoms and may be substituted by 0 to 6 halogen atoms, a hydroxyl group, an alkoxyl group having 1 to 6 carbon atoms, an alkylamino group having 1 to 6 carton atoms, a dialkylamino group having 1 to 6 carbon atoms, or an alkylthio group having 1 to 6 carbon atoms; an alkenyl group having 2 to 12 carbon atoms; an alkynyl group having 2 to 12 carbon atoms;
M may be two hydrogen atoms, a divalent metal, a monosubstituted trivalent metal, a disubstituted tetravalent metal, or an oxometal group;
S is a moiety containing at least one —SO₃; groups and
n is an integer from 1 to 4.

2-11. (canceled)

12. An optical recording material of claim 1 represented by formula II 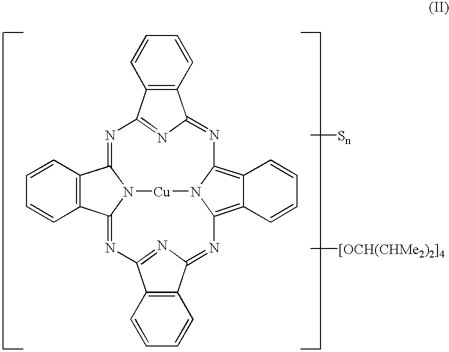
wherein each R₁, R₂, R₃, and R₄ is independently —CH(CHMe₂)₂.

13. An optical recording material of claim 12 represented by formula III 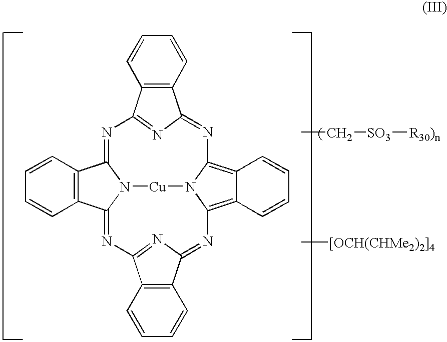
wherein S further comprises of —CH₂SO₃R₃₀, and R₃₀ is a trifluoromethyl group or a tolyl group.

14. A process for the manufacture of an optical recording material with structure represented by formula I 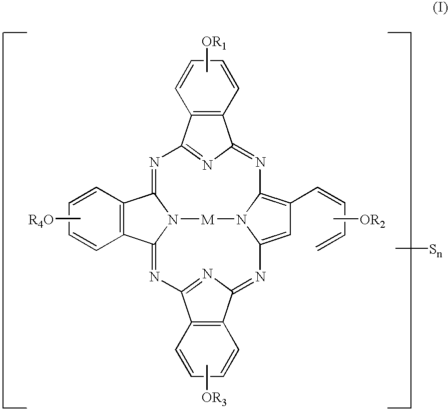
wherein
each R₁, R₂, R₃, and R₄ independently an alkyl group having 1 to 12 carbon atoms and may be substituted by 0 to 6 halogen atoms, a hydroxyl group, an alkoxyl group having 1 to 6 carbon atoms, an alkylamino group having 1 to 6 carbon atoms, a dialkylamino group having 1 to 6 carbon atoms, or an alkylthio group having 1 to 6 carbon atoms; an alkenyl group having 2 to 12 carbon atoms; an alkynyl group having 2 to 12 carbon atoms;
M may be two hydrogen atoms, a divalent metal, a monosubstituted trivalent metal a disubstituted tetravalent metal, or an oxometal group;
S is a moiety containing at least one —SO₃— group; and
n is an integer from 1 to 4;
comprising a step of covalently bonding a sulfonate compound to an unsubstituted or substituted phthalocyanine.

15. A process according to claim 14, further comprising a step of reacting a hydroxylated phthalocyanine with sulfonic anhydride or sulfonyl chloride under anhydrous conditions.

16. A process according to claim 15, wherein the sulfonic anhydride is a trifluoromethanesulfonic anhydride.

17. A process according to claim 15, wherein the sulfonyl choride is p-toluenesulfony chloride.

18. A process according to claim 17, wherein the reaction further contains an organic base as catalyst.

19. A process according to claim 18, wherein the organic base is pyridine.

20. A process according to claim 14, further comprising a step of reacting a sulfonyl chloride-substituted phthalocyanine derivative with an alcohol.

21. An optical recording medium comprising the optical recording material recited in claim 1.

22. An optical recording medium of claim 21, further comprising a substrate, a recording layer, a reflective layer, and protective layer, wherein the optical recording material is placed in the recording layer.

23. An optical recording medium comprising the optical recording material recited in claim 12.

24. An optical recording medium of claim 23, further comprising a substrate, a recording layer, a reflective. layer, and protective layer, wherein the optical recording material is placed in the recording layer.

25. An optical recording medium comprising the optical recording material recited in claim 13.

26. An optical recording medium of claim 25, further comprising a substrate, a recording layer, a reflective layer, and protective layer, wherein the optical recording material is placed in the recording layer.
